(19) United States
(12) Patent Application Publication
(10) Pub. No.: US 2014/0213970 A1
(43) Pub. Date: Jul. 31, 2014

(54) CATHETER BALLOON AND METHOD OF FABRICATION

(71) Applicant: Edwards Lifesciences Corporation, Irvine, CA (US)

(72) Inventors: Yidong M. Zhu, Irvine, CA (US); Marlowe E. Patterson, Orange, CA (US)

(73) Assignee: Edwards Lifesciences Corporation, Irvine, CA (US)

(21) Appl. No.: 14/098,078

(22) Filed: Dec. 5, 2013

Related U.S. Application Data
(60) Provisional application No. 61/757,617, filed on Jan. 28, 2013.

(51) Int. Cl. A61M 25/10; B29C 49/20; B29C 49/22; B29C 49/08

(52) U.S. Cl. CPC A61M 25/1002; A61M 25/1029; B29C 49/08; B29C 49/20; B29C 49/22
USPC 604/103.07; 264/515; 264/532

(57) ABSTRACT

A process for forming a catheter balloon includes subjecting a tubular parison in a mold to molding fluid pressure. The resulting catheter balloon includes a balloon portion having two ends and tubular leg portions extending from either end. The ends of the balloon portion are tapered to the tubular leg portions. The tapered ends have alternating elongate areas of greater and lesser resistance to deformation displaced circumferentially about the tapered ends. The elongate areas of greater and lesser resistance to deformation can include ridges which are either longitudinal or spiraled or rods imbedded in the body of the balloon. While in the mold, the tubular legs are drawn axially sufficiently to form permanent creases in the tapered ends. This drawing may be sufficient to cause the material of the tapered ends to exceed the yield strength, particularly with the balloon material in a malleable state.

### TECHNICAL FIELD

The present disclosure is directed to medical devices, and more particularly, to balloons for medical dilation procedures.

### BACKGROUND

Balloons associated with catheters for percutaneous procedures are now quite common. Such procedures include angioplasty, valvuloplasty, and urological dilation, which employ a balloon with or without companion equipment such as expandable stents to expand in the body. Percutaneous procedures can eliminate the need, risk and expense for more conventional surgical procedures and greatly reduce recovery time.

Balloons are employed with catheters which are thin, flexible lengths of tubing that are fed percutaneously through an arterial system to a location requiring wall or port expansion, or lining material compression. One or more balloons is appropriately placed along the length of the tubing, typically near the tip. The balloons are introduced in a contracted state for placement in a body passageway such as the lumen of a blood vessel, a urological passageway or the like. Fluoroscopic guidance typically assists in the appropriate threading and placement of the catheter and of the balloon mounted thereon. A guide wire typically extends from the distal end of the catheter and is able to move axially of the catheter to assist in the proper placement thereof. Further, sheaths are frequently concentrically arranged on the catheters and extend over the undeployed balloons. When the sheath is drawn axially from over the balloon, the balloon can then be expanded to a taut or optionally a distended state depending on the elasticity and other properties of the balloon material.

Once the balloon has been expanded to perform the appropriate procedure, the balloon is collapsed. Such a collapse can be provided by release of fluid pressure within the balloon, possible vacuum drawn on the balloon and/or re-extension of the sheath over the balloon. The extension of a sheath over the balloon is often difficult to accomplish, requiring an inconvenient level of force. Difficulties using a sheath are further compounded by the large scale of such devices used in percutaneous widening of a stenotic heart valve.

The balloons employed in such medical procedures are generally polymeric materials. Polyethylene terephthalates, polyvinyl chlorides, and cross-linked polyethylenes, as well as other materials, are known to be employed in the fabrication of balloons for percutaneous medical procedures.

These materials, whether non-distensible or distensible, are considered highly reliable, particularly in comparison with open chest cavity procedures, for example. Even so, failure concerns must be addressed. Catheter balloons have the possibility of tearing under load or manipulation. Such failure can occur either along a longitudinal or a circumferential tear. Longitudinal tears are considered relatively safe. Circumferential tears, on the other hand, are considered clinically unsafe. Retraction into a sheath can result in circumferential tears under adverse circumstances. However, increasing the wall thickness of such balloon materials increases the invasive aspect of the device. Thus, conflicting design criteria, particularly the diameter of the expanded balloon increases, must be reconciled.

Disclosures directed to existing balloon technology and balloon fabrication are found in U.S. Pat. Nos. 7,128,868; 6,500,148; 6,428,568; 5,350,361; and 5,147,302, the disclosures of which are incorporated herein by reference.

### SUMMARY

The present disclosure is directed to balloons for employment in percutaneous medical procedures. Mechanisms are employed for avoiding circumferential failures in use.

In a first separate aspect, a catheter balloon includes a tubular balloon portion with a tubular leg extending from each end. The balloon portion includes tapered ends which extend to the tubular legs. The tapered ends of the tubular balloon portion have alternating elongate areas of greater and lesser resistance to deformation, which elongate areas are circumferentially displaced about the tapered ends.

In a second separate aspect, a catheter balloon includes a tubular balloon portion with a tubular leg extending from each end. The balloon portion includes tapered ends which extend to the tubular legs. The tapered ends of the tubular balloon portion have alternating elongate areas of greater and lesser resistance to deformation, which elongate areas are circumferentially displaced about the tapered ends. The elongate areas of greater resistance include ribs in this separate aspect which may extend either longitudinally or in a spiral.

In a third separate aspect, a catheter balloon includes a tubular balloon portion with a tubular leg extending from each end. The balloon portion includes tapered ends which extend to the tubular legs. The tapered ends of the tubular balloon portion have alternating elongate areas of greater and lesser resistance to deformation, which elongate areas are circumferentially displaced about the tapered ends. The alternating elongate areas of greater resistance to deformation may be embedded rods which are positioned anywhere from the inside to the outside of the tubular body defining the balloon in this separate aspect.

In a fourth separate aspect, a process for forming a catheter balloon includes molding such a balloon from a tubular parison. The resulting balloon includes a balloon portion having two ends and tubular leg portions at either end of the balloon portion. At least one of the ends of the balloon portion is tapered to the respective tubular leg portion. The tapered end(s) are formed with elongate areas of greater and lesser resistance to deformation. The molded balloon is axially drawn partially from the mold in a manner sufficient to form permanent creases in one or both tapered ends.

In a fifth separate aspect, a process for forming a catheter balloon includes molding such a balloon from a coextruded tubular parison having rods embedded in the parison which are of material having greater resistance to deformation. The resulting balloon includes a balloon portion having two ends and tubular leg portions at either end of the balloon portion. At least one of the ends of the balloon portion is tapered to the respective tubular leg portion. The molded balloon is then axially drawn from the mold in a manner sufficient to form permanent creases in one or both tapered ends.

In a sixth separate aspect, any of the foregoing aspects are contemplated to be employed in combination to greater advantage.

Thus, it is a principal object to provide improved catheter balloons capable of resistance to circumferential tears and ease of resheathing. Other and further objects and advantages will appear hereinafter.

### BRIEF DESCRIPTION OF THE DRAWINGS

FIG. 1 illustrates a side view of a catheter balloon associated with a catheter with portions broken away for clarity.

FIG. 2 illustrates a cross-sectional side view of a catheter balloon in a forming mold.

FIG. 3 illustrates a second embodiment of a cross-sectional side view of a portion of a forming mold for a catheter balloon.

FIG. 4 illustrates a third embodiment of a cross-sectional side view of a portion of a forming mold for a catheter balloon.

FIG. 5 illustrates an end view of a first coextruded parison.

FIG. 6 illustrates an end view of a second coextruded parison.

FIG. 7 illustrates an end view of a third coextruded parison.

### DETAILED DESCRIPTION OF THE PREFERRED EMBODIMENTS

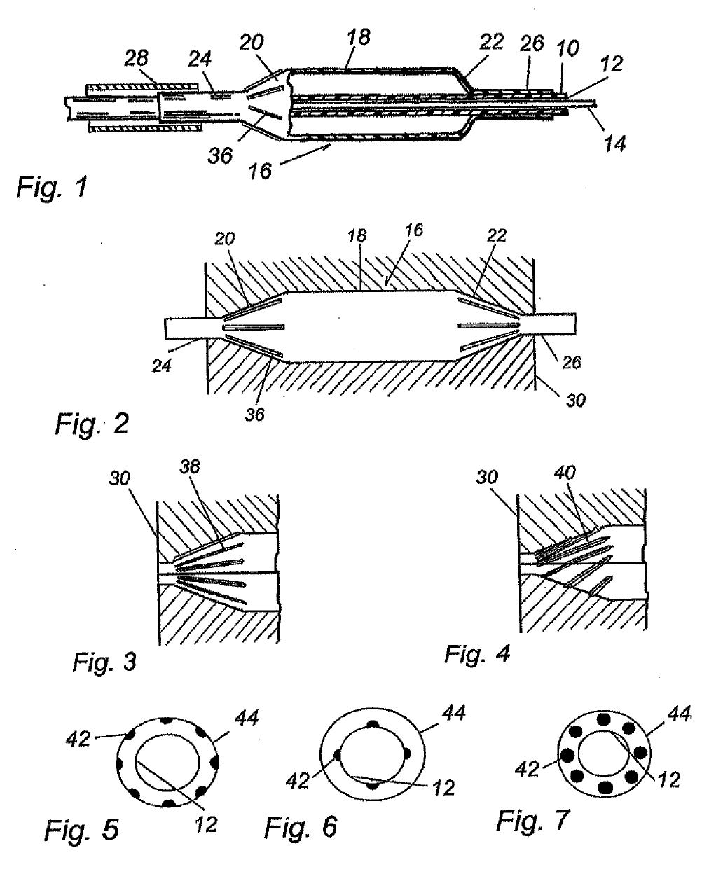

Turning in detail to the drawings, an inflated balloon associated with a catheter is illustrated in FIG. 1. The catheter 10, illustrated only in part, has a tube with a lumen 12 therethrough. A guide wire 14 extends through the lumen 12 to assist in placement of the catheter 10. The catheter also has at least one additional lumen, not shown, to feed pressurized fluid for balloon inflation. A balloon 16 is shown to be positioned on the catheter 10 and is sealed at both ends.

The balloon 16 includes a cylindrical balloon portion 18 with two ends 20, 22 which are tapered to tubular leg portions 24, 26. The entire balloon 16 has a passage therethrough with the balloon 16 having been molded from a tubular parison. In FIG. 1, the balloon 16 is shown in an inflated state.

Typically a balloon 16 is deployed percutaneously in a deflated state and also typically with an axially movable sheath 28 over the collapsed balloon 16. The sheath 28 is withdrawn from about the balloon 16 before inflation. The sheath 28 may be replaced over the balloon 16 before retraction of the catheter 10 from the vascular system. Balloon deflation precedes resheathing.

In fabricating the balloon 16 from a tubular parison, the balloon 16 is placed in a mold 30 such as roughly illustrated in FIG. 2. The mold 30 has the appropriate shape of the resulting balloon portion 18 with the tapered ends 20, 22 and passageways for the tubular leg portions 24, 26 and is split through the centerline of the resulting balloon 16. The tubular leg portions 24, 26 are actually unexpanded portions of the parison. Pressurized fluid is injected into the parison while in the mold cavity until the parison expands within the mold 30 to create the balloon 16. With release of the fluid, the mold 30 may be separated and the balloon 16 extracted.

The formed balloon 16 is designed to have alternating elongate areas of greater and lesser resistance to deformation circumferentially displaced about the two tapered ends 20, 22. FIG. 2 illustrates one embodiment of a balloon 16 with such circumferentially displaced elongate areas of greater resistance to deformation being in the form of ridges 36, shown in this example to be generally equiangularly displaced about the tapered ends 20, 22. Because of the added thickness afforded the tapered ends 20, 22 by the ridges 36, circumferential deformation and circumferential tearing are minimized. At the same time, folding is more likely constrained to the longitudinal areas of lesser resistance to deformation in between the ridges 36.

FIGS. 3 and 4 illustrate recesses 38 and 40, respectively, in the mold cavities in which the balloon 16 is to be fabricated. The recesses 38 illustrated in FIG. 3 extend longitudinally while the recesses 40 illustrated in FIG. 4 each extend in a spiral. In either case, the recesses 38, 40 receive material during the molding process forming the balloon 16 such that the ridges 36 remain after molding.

FIGS. 5, 6, and 7 illustrate another means to achieve alternating elongate areas of greater and lesser resistance to deformation circumferentially displaced about the two tapered ends 20, 22. In each case, the tubular parison is coextruded with rods 42 of material having greater resistance to deformation. The rods 42 are embedded in each case in the body of the parison 44. In FIG. 5, they are disposed about the periphery. In FIG. 6, they are disposed about the lumen 12. In FIG. 7, the rods 42 are displaced from both the lumen 12 and the outer periphery of the parison 44. The rods 42 may be of different but compatible material with the body of the parison 44 or simply be a denser or more cross-linked state of the same material. Various actual physical attributes may accomplish the appropriate result of increasing resistance to deformation. The rods 42 may be stiffer in bending. Alternatively, they may be tougher in resisting tearing or longitudinal extension. They also may simply have a different response to temperature.

In the case of the resistant rods 42, the most practical means for co-extruding the parison 44 is to have the rods 42 extend the full length of what ultimately becomes the balloon 16. Even so, it is understood to be principally the tapered ends 20, 22 which are most advantaged by the presence of these rods 42. When the balloon 16 is molded in the cavity 30, the entire body of the balloon 16, including the rods 42, moves radially outwardly to fill the mold cavity.

Once the balloon 16 has been configured in the molding process, one or both ends of the tubular leg portions 24, 26 can be axially pulled with the fluid pressure released and before the balloon 16 is removed from the mold 30. Pulling of the leg portions 24, 26 axially draws the balloon 16 partially from the mold 30. As the balloon 16 is moving into the tubular sections of the mold 30 reserved for the tubular leg portions 24, 26, the tapered ends 20, 22 are radially compressed. At the same time, there is a drawing action beyond the yield point of the tapered ends, 20, 22. This action may be undertaken with the balloon 16 still in a malleable state at a temperature determined by the type of material employed. The effect of this action is to create permanent creases extending longitudinally through part of or all of the tapered ends 20, 22. Once permanent creases 36 have been defined, the tubular leg portions 24, 26 are released and the mold 30 is opened. The presence of the rods 42, regardless of which location, for example, as exemplified in FIGS. 5-7, impacts the extension of the creases 36 formed. The creases 36 are understood to take the path of least resistance and would parallel the rods 42 to create longitudinal folds.

The intent of the creases 36 before deployment is to facilitate resheathing of the balloon 16 with a reduced force. The creases 36 provide a predisposition for the balloon 16 to appropriately fold and be drawn into the sheath 28. Through testing, it has been demonstrated that the amount of force required to resheath the balloon drops by in excess of one-half when such creases 36 are employed over uncreased balloons. This is understood to occur because the creases 36 are already formed in the material and less force is required to refold the permanently creased balloon 16. Depending upon the malleability of the material at the temperature in the mold 30, the creases 36 may extend more or less into the tapered ends 20, 22 and possibly even onto the cylindrical balloon portion 18. It is understood that the drawing of the balloon 16 from the tubular ends of the mold 30 results in an elongation of the tapered end section or sections 20, 22. As such, the mass per unit length decreases in this region or regions as compared with the portions of the balloon 16 where longitudinal extension does not occur.

With any of the foregoing embodiments, folding of the balloon 16 to assemble the catheter 10 with the sheath 28 before use is undertaken. Attention is paid to the creases 36 that have been defined in the tapered end portions 20, 22 of the balloon portion 18 in that process.

Thus, an improved catheter balloon 16 having permanent creases 36 to reduce the force required in resheathing after expansion has been disclosed. Further, alternating elongate areas of greater and lesser resistance to deformation are understood to inhibit circumferential splitting of the balloon 16. While embodiments and applications have been shown and described, it would be apparent to those skilled in the art that many more modifications are possible without departing from the concepts disclosed herein. The disclosure, therefore, is not to be restricted except in the spirit of the appended claims.

What is claimed is:

1. A catheter balloon comprising:
a tubular balloon portion having two tapered ends with alternating elongate areas of greater and lesser resistance to deformation circumferentially displaced about the two tapered ends; and
a tubular leg extending from each of the tapered ends.

2. The catheter balloon of claim 1, the alternating elongate areas of greater and lesser resistance to deformation being ridges angularly displaced from one another on an exterior of the two tapered ends.

3. The catheter balloon of claim 2, the ridges extending longitudinally.

4. The catheter balloon of claim 2, each ridge extending in a spiral.

5. The catheter balloon of claim 1, the alternating elongate areas of greater and lesser resistance to deformation being rods imbedded in and extending from one tubular leg to the other tubular leg through the tubular balloon portion and being angularly displaced from one another.

6. The catheter balloon of claim 5, the rods being at an inner surface of the tubular legs and the tubular balloon portion.

7. The catheter balloon of claim 5, the rods being at an outer surface of the tubular legs and the tubular balloon portion.

8. The catheter balloon of claim 5, the rods being displaced from both inner and outer surfaces of the tubular legs and the tubular balloon portion.

9. A process for forming a catheter balloon comprising the steps of:
molding a catheter balloon in a mold from a tubular parison, the molded catheter balloon including a balloon portion having two ends and tubular leg portions at either end of the balloon portion, at least one of the ends of the balloon portion being tapered to the respective tubular leg portion with alternating elongate areas of greater and lesser resistance to deformation circumferentially displaced about the at least one tapered end;
axially drawing the molded catheter balloon partially from the mold at least at one end sufficiently to form permanent creases in the at least one tapered end.

10. The process for forming a catheter balloon of claim 9, the step of molding being from a tubular parison coextruded with rods of material having greater resistance to deformation.
